(19) United States
(12) Patent Application Publication
(10) Pub. No.: US 2023/0174449 A1
(43) Pub. Date: Jun. 8, 2023

(54) METHOD FOR RECOVERING HEAVY BY-PRODUCTS FROM ACRYLIC ACID AND ESTERS OF SAID ACID BY THERMAL CRACKING WITH PARTIAL CONDENSATION

(71) Applicant: Arkema France, Colombes (FR)

(72) Inventors: Michel FAUCONET, Saint Avold cedex (FR); Frederic SANDRE, Saint Avold cedex (FR)

(21) Appl. No.: 17/922,984

(22) PCT Filed: May 11, 2021
(86) PCT No.: PCT/FR2021/050825

§ 371 Date: Nov. 3, 2022

(30) Foreign Application Priority Data
May 19, 2020 (FR) FR2005057

(51) Int. Cl. C07C 51/377; C07C 51/487; C07C 67/54; C07C 67/60; C07C 51/44; C07C 67/327

(52) U.S. Cl. CPC C07C 51/377; C07C 51/487; C07C 67/54; C07C 67/60; C07C 51/44; C07C 67/327

(57) ABSTRACT

The present invention relates to an improved process for the regeneration, by thermal cracking, of a mixture of heavy byproducts (residues) resulting from a unit for the production of acrylic acid and from a unit for the production of acrylic ester, resulting in acrylic acid, acrylic esters and alcohols being obtained, for the purpose of recycling them in the plant for the production of the acrylic ester.

### FIELD OF THE INVENTION

The present invention relates to an improved process for the regeneration, by thermal cracking, of a mixture of heavy byproducts (residues) resulting from a unit for the production of acrylic acid and from a unit for the production of acrylic ester, resulting in acrylic acid (AA), acrylic esters (AE) and alcohols being obtained, for the purpose of recycling them in the plant for the production of the acrylic ester.

### TECHNICAL BACKGROUND

Under the effect of the temperature during the distillation stages, the manufacture of acrylic acid and of acrylic acid esters is accompanied by the formation of heavy compounds, derivatives of the addition of compounds having a nucleophilic property to the double bond of the unsaturated carbonyl-containing monomers, by the Michael reaction. “Heavy” compound refers to the compounds having a greater boiling point than that of the acrylic monomer manufactured.

The main factor limiting the efficiency of the regeneration of the compounds derived from the Michael reaction contained in the heavy streams from AA and acrylic ester plants is the increase in the viscosity of the heavy residue obtained at the cracker bottom, when the fraction rich in acrylic monomers and alcohol generated by the cracking has been evaporated.

The evaporation of light compounds during the cracking results in a concentration of the heavy products in the residue stream and an increase in the viscosity of this stream. However, the residue has to remain sufficiently fluid after cooling to be transported and then treated for the purpose of destroying it.

In the document EP 717 031, it has been shown that it is possible to improve the efficiency of the recovery of these upgradable noble products, if the cracking is carried out starting from a mixture of heavy products originating from a unit for the production of AA and from a unit for the production of acrylic ester, in comparison with the individual cracking of the heavy streams from these units. The effect of the addition of heavy products originating from the ester units to the heavy products originating from an AA unit is to reduce the viscosity of the final residue. The cracking reaction is carried out starting from mixtures having an AA heavy products/ester heavy products ratio of 9/1 to 1/9, at a temperature of 180 to 220° C., under atmospheric pressure, for a residence time of 0.5 to 3 hours. In this process, the cracking and the evaporation of the light compounds generated are carried out in a reactor, then the gas stream generated is sent into a distillation column and, finally, the bottom stream from the distillation column is recycled to the reactor. On the other hand, as the light fraction obtained by cracking consists mainly of AA and ester acrylic monomers, which are particularly sensitive to polymerization, the distillation stage necessarily has to be carried out under reduced pressure, so as to reduce the temperature, in order to prevent the formation of polymer in the column Furthermore, the rectifying plates of the distillation column bring about the efficient separation of the polymerization inhibitors entrained in the gas mixture, which flow back to the column bottom, and consequently it is necessary to introduce fresh polymerization inhibitors at the column top, in order to prevent the formation of polymers in the upper part of the column. For this reason, the reaction stage, carried out under higher pressure, and the distillation stage, carried out under reduced pressure, have to be separated. The installation for carrying out the process thus has to be equipped with a reactor and with a top condenser, which are operated at the same pressure, and with a distillation column operated at reduced pressure, fed with the condensed product, and comprising a boiler at the bottom and, at the top, a condenser, an item of reflux equipment and a feed of inhibitors. This arrangement is complicated and expensive.

As a result of the presence of a high concentration of alcohol in the acrylic ester synthesis streams, a major part of the streams of heavy products from these production units consists of derivatives of the addition of the alcohol to the AA and monomer ester double bond. For example, in the case of ethyl acrylate, they are ethyl 3-ethoxypropionate and 3-ethoxypropionic acid. These alkoxy-propionic compounds comprise C—O—C bonds which are more difficult to break than the C—C bonds of the other derivatives. Under the temperature conditions normally used for the regeneration of acrylic heavy products, they can only be dissociated in the presence of catalyst at high concentration. For this reason, the amount of products which can be upgraded starting from heavy products from acrylic ester units is lower than that from acrylic acid units. The amount of compounds which can potentially be upgraded by thermal cracking is thus approximately two times greater in the heavy products from an AA unit. In the platforms for the production of acrylic monomers, there consequently exists an advantage in upgrading, as a priority, the heavy products from an AA unit, in comparison with the heavy products from ester units.

However, when the AA heavy products/AE heavy products (AAHP/AEHP) ratio increases, the increase in the viscosity of the residue from the cracking reaction is increasingly sensitive to the parameters making it possible to improve the cracking yield, namely an increase in the reaction temperature and/or an increase in the reaction time. This effect is particularly important when the ratio of AA unit heavy products with respect to the combined mixture of AA heavy products and ester heavy products exceeds 50%.

Consequently, there exists a need to improve the yield of regeneration, by thermal cracking, of the heavy compounds resulting from the AA and ester units containing more than 50% of AA heavy products, while decreasing the effect of increase in viscosity of the residue during the evaporation of the monomer-rich mixture during the cracking.

As has been seen above, the efficiency of the regeneration depends essentially on two parameters: the temperature and the residence time of the reaction. The increase in these two parameters tends to improve the regeneration yield but this takes place to the detriment of an increase in the viscosity of the cracking residue.

In order to efficiently crack Michael derivatives contained in the AAHP and AEHP mixtures, a minimum temperature of approximately 140° C. is necessary but, at this temperature, the reaction time has to be very long if it is desired to obtain an acceptable yield. In the specific case of the thermal dissociation of Michael derivatives starting from mixtures of AA and AE heavy products, an increase in the residence time at this relatively low temperature level is not favorable for increasing the recovery yield because there exists a competition between the cracking reaction of the Michael derivatives to give monomers and that of formation of the Michael derivatives from the monomers, the latter being favored by the intermediate temperatures (140-160° C.) and the high reaction times. It is thus preferable to carry out the cracking at a higher temperature (above 160° C., preferably at more than 180° C.) and at a lower residence time (less than 6 h, preferably less than 4 h).

In the platforms for the production of AA and AE, it is very usual to have large fluctuations in flow rates of production of AA and AE, depending on market demands, and correspondingly fluctuations in flow rates of heavy byproducts from these units. Consequently, when the cracking operation is carried out by a process in continuous mode, strong fluctuations may be observed in the residence time in the cracking reactor, which depends on the feed flow rate and on the ratio of heavy products originating from the AA and ester units.

There consequently exists an advantage in having available a process for the cracking of heavy compounds from AA and AE units which makes it possible to obtain good upgrading yields, whatever the fluctuations in flow rates and in ratios of AA heavy products and AE heavy products.

### SUMMARY OF THE INVENTION

The invention relates to a process for the regeneration of a mixture of heavy byproducts originating from a unit for the production of acrylic acid and of heavy byproducts originating from a unit for the production of acrylic ester of formula CH₂═CH—C(═O)—OR, R being a C₁-C₈ alkyl group, said process comprising the following stages:
i. introducing said mixture into a reactor and subjecting it to a thermal cracking, producing a gaseous top stream and a bottom stream (residue) concentrated in heavy products,
ii. subjecting said top stream to a partial condensation, resulting in a top stream being obtained containing acrylic acid, acrylic ester and alcohol R—OH, which stream is intended in particular for recycling to the acrylic ester production plant, and in a bottom stream being obtained,
iii. recovering said residue for the purpose of a removal treatment.

Advantageously, stages i) and ii) are carried out at the same absolute pressure, of between 200 and 2000 hPa, preferably between 300 and 1500 hPa.

According to various implementations, said process comprises the following characteristics, if appropriate combined.

According to one embodiment, the partial condensation operation is carried out in an exchanger (condenser) bringing about the condensation of a portion of the vapor generated during the thermal cracking, without this vapor being subjected beforehand to a fractionation in a distillation column containing one or more rectifying plates.

According to one embodiment, the temperature of the gas mixture (top stream) exiting from the partial condensation stage is of between 140° C. and 180° C.

According to one embodiment, the cracking temperature is of between 140° C. and 260° C., preferably between 180° C. and 210° C.

According to one embodiment, the residence time of the reaction mixture in the cracking reactor is of between 0.5 h and 10 h, preferably between 2 h and 7 h.

According to one embodiment, the bottom stream from the reactor (residue) obtained on conclusion of the thermal cracking operation exhibits a dynamic viscosity of less than 1 Pa·s, measured at a temperature of 100° C. for example using a Brookfield “CAP 1000+” viscometer of cone/plate type.

According to one embodiment, the bottom stream from the partial condenser is at least partially recycled to the reactor.

According to one embodiment, the liquid bottom stream from the partial condenser is recycled directly to the liquid phase of the reactor, via a dip pipe in the reactor, or to a pipe for recirculation of the reaction medium through an external exchanger, preferably upstream of this external exchanger.

According to one embodiment, the noncondensed gaseous top stream resulting from the partial condenser, rich in AA, acrylic ester and alcohol, is recycled to a unit for the manufacture of acrylic ester, preferably in liquid form, after condensation in a second condenser.

According to one embodiment, the ratio by weight of condensed stream to gas stream entering the condenser is of between 5% and 50%, preferably between 10% and 40%.

According to one embodiment, the AAHP/AEHP ratio by weight is of between 3/7 and 9/1, preferably between 1/1 and 9/1.

According to one embodiment, said radical R is a C₁-C₄ alkyl group.

According to one embodiment, the ester is ethyl acrylate and the alcohol is ethanol.

According to one embodiment, said regeneration process is carried out continuously.

According to one embodiment, polymerization inhibitors are introduced at the partial condenser and, when it is employed, at the second condenser. These inhibitors are chosen from polymerization inhibitors known to a person skilled in the art: phenol derivatives, such as hydroquinone and its derivatives, for example hydroquinone methyl ether, 2,6-di(tert-butyl)-4-methylphenol (BHT) and 2,4-dimethyl-6-(tert-butyl)phenol (Topanol A), phenothiazine and its derivatives, manganese salts, such as manganese acetate, salts of thiocarbamic or dithiocarbamic acid, such as metal thiocarbamates and dithiocarbamates, for example copper di(n-butyl)dithiocarbamate, N-oxyl compounds, such as 4-hydroxy-2,2,6,6-tetramethylpiperidineoxyl (4-OH-TEMPO), compounds having nitroso groups, such as N-nitrosophenylhydroxylamine and its ammonium salts, amine compounds, such as para-phenylenediamine derivatives, or a mixture of these inhibitors.

According to one embodiment, the thermal cracking reaction takes place in the absence of catalyst.

According to one embodiment, the thermal cracking reaction takes place in the presence of catalysts. Mention may be made, as catalysts, of Broensted acids, such as sulfuric acid, sulfonic acids, phosphoric acid, zeolites, aluminas or catalysts based on silica and alumina, Lewis acids, such as, for example, titanates, bases, alkali metals or their salts, amines, phosphines, or their combination.

The present invention makes it possible to overcome the disadvantages of the state of the art. It provides more particularly a simplified process for the thermal cracking of a mixture of heavy byproducts originating from a unit for the production of acrylic acid and of heavy byproducts originating from a unit for the production of acrylic ester, making it possible to efficiently recover monomers which can be upgraded in the manufacture of the acrylic ester. This is accomplished by virtue of the combination of a stage of thermal cracking of said mixture and of a stage of partial condensation of the top stream resulting from the cracking, the two stages being carried out at the same pressure.

Advantageously, the process for the cracking of heavy compounds from AA and AE units according to the invention makes it possible to obtain good upgrading yields, whatever the fluctuations in flow rates and in ratios of AA heavy products and AE heavy products.

The main advantages of the process according to the invention are:
An increase in recovery yield of noble products from the heavy products derived from the Michael reaction present in the streams of heavy products from AA and ester plants, by more thoroughly forcing the cracking reaction, without limit because of the viscosity, and by treating mixtures of AA heavy products and ester heavy products richer in AA heavy products;
A process which is simple and relatively inexpensive in capital costs since it requires virtually only one additional item of equipment (the partial condenser) and which is simple to carry out (partial condensation stage at the same pressure as the cracking reaction);
A process which makes it possible to reduce the discharges by decreasing the amount of cracking residue;
A process which makes it possible to optimize the recovery yield, despite the fluctuation in the residence time and in the AA heavy products/ester heavy products ratio.

### FIGURES

FIG. 1 diagrammatically represents an embodiment of an installation according to the invention.

FIG. 2 diagrammatically represents another embodiment of an installation according to the invention.

### DESCRIPTION OF EMBODIMENTS OF THE INVENTION

The invention is now described in more detail and in a non-limiting way in the description which follows.

The term “heavy byproducts originating from a unit for the production of acrylic acid” comprises:
derivatives of the addition of acrylic acid to the double bond of another acrylic acid molecule: 3-acryloyloxypropionic acid, also called “acrylic acid dimer” or “AA dimer”;
derivatives of the addition of acrylic acid to the double bond on an AA dimer molecule, to form the “AA trimer”, and other oligomers formed by successive additions of acrylic acid to the double bonds of the preceding AA oligomers;
derivatives of the addition of carboxylic acid formed as byproducts of acrylic acid (for example acetic acid) or of water to the double bond of the AA or of the abovementioned oligomers.

The term “heavy byproducts originating from a unit for the production of acrylic acid ester (AE)” comprises the same compounds as those mentioned above, and also:
the alkyl esters of these compounds, the alkyl radical being that of the AE ester;
the derivatives of the Michael reaction addition resulting from the addition of alcohol to the double bonds of the acrylic acid, of the AA dimer or of the AA oligomers, and also their esters.

The term “partial condensation” refers to an operation during which the gas stream generated during the thermal cracking stage is partially condensed in the form of a liquid stream.

The term “partial condenser” refers to an item of equipment making it possible to generate a liquid fraction (reflux) from a hot vapor stream, by cooling on a wall which is colder than the vapors. It is generally a heat exchanger kept colder than the vapors by the circulation of a refrigerant. The refrigerant can, for example, be a gas stream, such as air, water, or any other liquid stream originating from a supply external to the process or recycled from a stream of the process. The temperature of the vapors at the outlet of the exchanger is regulated at the temperature desired in order to obtain a greater or lesser stream of liquid by partial condensation, by modifying the temperature and the flow rate of the refrigerant in contact with the wall in the partial condenser. Mention may be made, as types of heat exchangers, of shell-and-tube exchangers, consisting of a shell and of a bundle of tubes within, in which the two fluids (vapors generated in the reactor and refrigerant) circulate separately. Mention may also be made of spiral exchangers or plate exchangers, or any other type of exchanger known to a person skilled in the art which makes possible the partial condensation of vapors. These exchangers can be horizontal or vertical items of equipment.

The terms of “total condensation” or “total condenser” refer to an operation or item of equipment carrying out a condensation of the condensable compounds contained in the non-condensed vapors at the outlet of the partial condenser, in the form of a liquid (distillate) which is withdrawn.

The invention is based on a thermal cracking process, coupled to a partial condensation operation, the two stages being carried out at the same pressure. The process of the invention applies to the regeneration of acrylic monomers from a mixture of heavy byproducts originating from a unit for the production of acrylic acid and of heavy byproducts originating from a unit for the production of acrylic ester, and makes it possible to efficiently recover these monomers which can be upgraded in the manufacture of the acrylic ester.

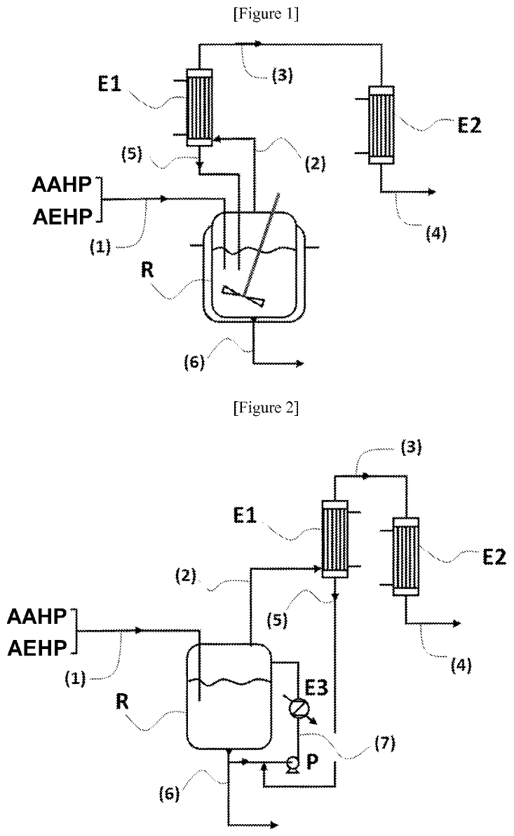

According to the embodiment of the process represented in FIG. 1, a mixture of streams originating from plants for the production of acrylic acid (AAHP) and of acrylic ester (AEHP) is introduced continuously in the liquid form into the reactor R. This mixture (1) is rich in heavy compounds derived from the Michael addition which are generated during the stages of synthesis and purification of the corresponding monomers (AA and AE), and also contains other heavy compounds accumulated during the processes of synthesis and purification, in particular polymerization inhibitors. The mixture (1) is introduced into the reactor, where it is heated to the temperature required to carry out the cracking of the derivatives of the Michael addition to give lighter compounds which are extracted in the form of a gas mixture (2) at the reactor top. This gas stream is sent to a partial condenser E1 consisting, for example, of a shell-and-tube exchanger. Said exchanger is circulated with water at a temperature and with a flow rate which are suitable for cooling the vapor to the required temperature, measured in the noncondensed vapor stream (3) at the outlet of the exchanger, and for obtaining the desired condensation ratio. This noncondensed vapor stream rich in upgradable compounds (AA, ester, alcohol) and containing a few heavy compounds in a low concentration is advantageously recycled to the process for the production of the AE ester, either directly in the vapor form or after total condensation in an exchanger E2. In the latter case, it is a liquid stream (4) which is recycled to the AE ester production unit.

At the bottom of the partial condenser, there is recovered a liquid stream (5) (also called reflux) richer in heavy compounds, in particular 3-alkoxypropionic Michael derivatives, the boiling point of which is intermediate between that of the monomers and that of the heaviest compounds, and also containing a small amount of polymerization inhibitors. This stream (5) is returned, at least partially, to the reactor, by introducing it preferably directly into the liquid phase of the reactor, via a separate dip pipe or via the dip pipe for feeding the reactor with AAHP and AEHP compounds.

According to a form not described in FIG. 1, a portion of the liquid stream condensed in the partial condenser, containing polymerization inhibitors at a low concentration, is returned using a pump to the top outlet of the partial condenser, so as to protect the item of equipment against a risk of polymerization within the condenser.

The residue stream recovered at the reactor bottom (6) is cooled and then removed in the form of a liquid of moderate viscosity, so as to be able to be transported without difficulty by pump, for example as far as a storage tank or an incineration unit.

According to an embodiment not described in FIG. 1, the feed stream composed of AAHP and AEHP is preheated through a heat exchanger before being introduced into the reactor.

According to another embodiment, the thermal cracking reaction is carried out noncontinuously.

According to one embodiment, the feed stream is introduced directly into the liquid phase of the reactor, via a dip pipe.

The diagram of FIG. 2 describes an embodiment of the process according to the same principle as that of FIG. 1, specifying a possible implementation in the case where the reactor is heated by recirculation, using a pump P, of the reaction medium contained in the reactor (7) through a heat exchanger E3. In this embodiment, the liquid stream faction (5) returned to the reactor will advantageously be recycled in the recirculation loop, preferentially upstream of the exchanger E3, for example upstream of the recirculation pump P.

### EXAMPLES

The following examples illustrate the invention without limiting it.

With reference to the instructions of FIG. 1, in these examples, the reaction temperature is the temperature measured in the liquid contained in the liquid phase of the reactor R, the “top temperature” is the temperature of the vapors measured at the outlet of the partial condenser E1 (stream 3), the “residence time” in the reactor is measured by the ratio by weight of the working volume of the reactor (volume of liquid phase in the reactor) to the feed flow rate of the heavy products (stream 1). The “topping ratio” is the ratio by weight of the condensed stream flow rate at the outlet of the total condenser E2 (stream 4) to the feed flow rate (stream 1), the “useful recovery rate” is the ratio by weight of the sum of the upgradable compounds (AA, AE and ethanol) collected in the distillate after total condensation (stream 4) to the feed flow rate of heavy products (stream 1). The “useful conversion rate” is the ratio by weight of the sum of the upgradable compounds (AA, AE and ethanol) which are generated by cracking and recovered in the totally condensed stream (stream 4), after subtraction of the sum of these compounds present in the feed (stream 1), with respect to the feed flow rate of the reactor (stream 1), after subtraction of the upgradable compounds present in this stream. Finally, the “viscosity @100° C.” is the value of the viscosity in Pa·s at a temperature of 100° C. of the residue obtained at the reactor bottom (stream 6), measured using a Brookfield “CAP 1000+” viscometer of cone/plate type, equipped with the No. 2 spindle, at a rotational speed of 750 rotations per minute.

All the tests were carried out under atmospheric pressure.

### Example 1: Cracking with Partial Condensation and Reflux of the Condensed Liquid into the Gaseous Headspace of the Reactor

The assembly consists of a glass reactor with a total volume of 1 liter, heated by recirculation of oil through its jacket, equipped with a side outlet for the exit of the residue, providing a working volume of liquid contained in the reactor of 240 ml.

The reactor is equipped with a stirrer, with a temperature probe immersed in the liquid phase, with a vertical pipe in the upper part, for the extraction of the vapors, and with a partial condenser. The partial condensation is provided by cooling of the reaction vapors on a thimble placed inside the vertical tube for extraction of the vapors, in which oil circulates at a regulated temperature of 150° C. A temperature probe makes it possible to measure the temperature of the noncondensed vapors at the outlet of the partial condenser. These noncondensed vapors are directed to a total condenser, consisting of a cooling apparatus provided with a jacket in which water circulates at 10° C. The liquid (distillate) is recovered in a receiver and analyzed. The assembly elements in contact with the vapors, from their generation in the reactor up to their entry into the second condenser, are lagged in order to reduce heat exchanges with the outside. In this assembly, the vapor fraction condensed in the partial condenser naturally flows back to the reactor.

A mixture consisting of 60% by weight of heavy compounds originating from a unit for the production of AA and 40% by weight of heavy compounds originating from a unit for the production of ethyl acrylate (EA) is sent continuously into the reactor, with a flow rate of 70 g/h. Under these conditions, the residence time is 3.4 h. The feed mixture is composed essentially of upgradable compounds (9% AA and less than 0.1% of AE and of ethanol), of heavy compounds derived from the Michael reaction (35.5% of AA dimer (AA2), 1.5% of AA trimer (AA3), 3.3% of 3-ethoxypropionic acid (EPA), 0.3% of 3-hydroxypropionic acid (HPA), 6.2% of ethyl 3-ethoxypropionate (EEP), 6.7% of ethyl 3-acryloyloxypropionate (EAP), 0.5% of ethyl 3-hydroxypropionate (EHP), and polymerization inhibitors (2.7% of hydroquinone (HQ), 0.6% of phenothiazine (PTZ)).

In example 1, after operation under stable conditions for more than 24 h, the reaction temperature is 195° C. and the temperature of the vapors at the outlet of the partial condenser is 157° C.

The operating conditions and performance qualities of these tests are summarized in table 1.

### Examples 2 and 3 (Comparative): (SF57-4 and SF58-4) Cracking without Condensation, According to the Prior Art

These two experimental examples were carried out in the same assembly as example 1 and under identical conditions, apart from the fact that the internal thimble is empty, which suppresses the effect of cooling by circulation of the refrigerant.

After operation under stable conditions for more than 24 h, the results obtained are described in table 1 below.

TABLE 1

| | |
|---|---|
| According to | |
| the invention | Comparative |

| Test No. | 1 | 2 | 3 |
|---|---|---|---|
| Residence time | 3.5 h | 3.5 h | 6 h |
| Reaction temperature | 195° C. | 194° C. | 187° C. |
| Top temperature | 157° C. | 177° C. | 168° C. |
| Topping ratio | 58.4% | 61.1% | 57.8% |
| Useful recovery rate | 43.2% | 41.7% | 37.3% |
| Useful conversion rate | 37.6% | 35.9% | 31.0% |
| Viscosity of the residue | 0.318 | 0.451 | 0.380 |
| in Pa · s @ 100° C. | | | |

These comparative examples show the advantage of a decrease in the temperature of the vapor at the reaction outlet resulting in the partial condensation of this vapor and a liquid reflux into the reactor, the consequence being an improvement in the recovery yield of noble compounds, with a reduced viscosity of the residue from the cracking reaction.

It is also observed that the reaction temperature of test 3, carried out with a residence time of 6 h, in comparison with test 2, carried out with a residence time of 3.5 h, is much lower, to obtain a similar cracking residue. At higher temperature, the viscosity of the residue increases strongly and makes it difficult to transport the liquid. The consequence of the decrease in temperature which can be used at a higher residence time without excessive increase in viscosity is a major reduction in the cracking performance qualities.

### Example 4: Partial Condensation of a Stream Resulting from a Reaction for the Cracking of AA Heavy Products and AE Heavy Products

A liquid mixture, originating from the total condensation of an operation for the cracking of a mixture of AAHP (60%) and of AEHP (40%), is vaporized and partially condensed in a shell-and-tube condenser, at a temperature of 160° C. A condensed liquid stream is obtained which represents 30% by weight of the feed stream of the condenser. This liquid condensate has a composition representative of the reflux (5) of FIG. 1, containing 35.4% of AA, 0.3% of AE, 4.4% of AA2, 0.4% of AA3, 11.5% of EPA, 22% of EEP, 6.2% of EAP, 0.1% of EHP, 0.3% of HQ, 0.04% of PTZ.

### Examples 5 to 8: Cracking with Partial Condensation and Recycling of the Condensed Stream into the Liquid Phase of the Reactor

These examples are intended to illustrate the operating conditions of the process according to the invention of FIG. 1. The operating conditions and performance qualities of these different tests are summarized in table 2.

In addition to the definitions described above, the “useful conversion rate” is the ratio by weight of the sum of the upgradable compounds (AA, AE and ethanol) which are generated by cracking and recovered in the totally condensed stream (stream 4), after subtraction of the sum of these compounds present in the feed (stream 1) and in the recycled stream (stream 5), with respect to the feed flow rate of the reactor (stream 1), after subtraction of the upgradable compounds present in this stream (1).

The experimental assembly is the same as that of example 1, apart from the fact that the vapors generated during the cracking reaction are sent directly to the total condenser (E2) and a flow rate of condensate stream obtained in example 3 is introduced by dip pipe into the reactor, in addition to the feed of AAHP and AEHP. This arrangement makes it possible to rigorously simulate the recycling of the cracker stream condensed at a temperature of 160° C., according to the expected recycling flow rate/feed flow rate of AAHP and AEHP ratio of 22% corresponding to a rate of condensation of the vapors of 30%.

In examples 5, 6 and 7 (residence time of 3 h), the reactor is fed with the mixture of AAHP and AEHP described in example 1, at a flow rate of 80 g/h, and the condensate stream of example 3 (stream 5) is introduced into the reactor, by dip pipe into the liquid phase, at a flow rate of 17.6 g/h. In example 8 (residence time of 6 h), the feed flow rate (1) of AAHP and AEHP is 40 g/h and the recycled condensate flow rate (5) is 8.8 g/h.

After operation under stable conditions for more than 24 h, the samples of streams resulting from the total condenser E2 (stream 4) and of reactor bottom residue (stream 6) are withdrawn and analyzed.

### Examples 9 and 10 (Comparative): Cracking without Condensation, According to the Prior Art

The assembly used and the operating conditions are the same as those described in examples 5 to 8, apart from the fact that the recycling of stream resulting from a partial condensation (stream 5) has been eliminated. As for examples 5 to 7, the feed flow rate of AAHP and AEHP of example 9 (residence time of 3 h) is 80 g/h, and that of example 10 (residence time of 3 h) is the same as that of example 8, i.e. 40 g/h.

The operating conditions and performance qualities of these tests are summarized in table 2 below.

TABLE 2

| | According to the invention | | | | Comparative | |
|---|---|---|---|---|---|---|
| Test No. | 5 | 6 | 7 | 8 | 9 | 10 |
| Residence | 3 h | 3 h | 3 h | 6 h | 3 h | 6 h |
| time | | | | | | |
| Reaction | 200° C. | 204° C. | 207° C. | 194° C. | 196° C. | 186° C. |
| temperature | | | | | | |
| Topping | 60.6% | 64.9% | 68.7% | 65.8% | 67.5% | 54.2% |
| ratio | | | | | | |
| Useful | 47.8% | 51.3% | 53.1% | 48.3% | 44.9% | 37.2% |
| recovery rate | | | | | | |
| Useful | 45.6% | 49.8% | 52.0% | 46.2% | 38.3% | 29.7% |
| conversion rate | | | | | | |
| Viscosity of the | 0.064 | 0.042 | 0.090 | 0.107 | 0.499 | 0.132 |
| residue in | | | | | | |
| Pa · s @ | | | | | | |
| 100° C. | | | | | | |

These examples show that it is possible to significantly improve the recovery yield of the upgradable products by carrying out a partial condensation of the cracking vapors and by recycling the condensed part to the reactor, in a very simple assembly employing a condenser operated at the same pressure as the cracking reaction, while obtaining a lower viscosity of the cracking residue than in the prior art.

Tests 9 and 10 make it possible to compare the cracking performance qualities at different residence times. For an equivalent viscosity of the residue, the comparison of tests 7 and 8 shows that the cracking reaction temperature has to be reduced when the residence time is higher, but the recovery performance qualities remain all the same much better under these conditions of higher residence time (test 8) than in test 9, which was carried out according to the prior art under nevertheless more favorable operating conditions of shorter residence time, with a viscosity of the residue which is, in addition, lower. This thus shows that the process according to the invention makes it possible to continue to recover much more noble product from one and the same AAHP-AEHP mixture, even when the flow rate of heavy products to be treated is lower for reasons of fluctuation in the rates of production of the AA and ester plants which result in an increase in the residence time.

1. A process for the regeneration of a mixture of heavy byproducts originating from a unit for the production of acrylic acid and of heavy byproducts originating from a unit for the production of acrylic ester of formula CH₂═CH—C(═O)—OR, R being a C₁-C₈ alkyl group, said process comprising the following stages:
i. introducing said mixture into a reactor and subjecting it to a thermal cracking, producing a gaseous top stream and a bottom stream residue concentrated in heavy products,
ii. subjecting said top stream to a partial condensation, resulting in a top stream being obtained containing acrylic acid, acrylic ester and alcohol R—OH and in a bottom stream being obtained,
iii. recovering said residue for a purpose of a removal treatment.

2. The process as claimed in claim 1, in which stages i) and ii) are carried out at a same absolute pressure.

3. The process as claimed in claim 1 in which a cracking temperature is between 140° C. and 260° C.

4. The process as claimed in claim 1 in which a residence time of the reaction mixture in a cracking reactor is of between 0.5 h and 10 h.

5. The process as claimed in claim 1 in which the bottom stream from the reactor (residue) obtained on conclusion of the thermal cracking operation exhibits a dynamic viscosity, measured at 100° C., of less than 1 Pa·s.

6. The process as claimed in claim 1 in which the bottom stream from a partial condenser is at least partially recycled to the reactor.

7. The process as claimed in claim 1 in which said top stream resulting from the partial condenser and containing acrylic acid, acrylic esters and alcohols R—OH is recycled to a unit for the manufacture of acrylic ester.

8. The process as claimed in claim 1 in which said top stream resulting from the partial condenser and containing acrylic acid, acrylic esters and alcohols R—OH is recycled to a unit for the manufacture of acrylic ester, in liquid form, after condensation in a second condenser.

9. The process as claimed in claim 1 in which the ester is ethyl acrylate and the alcohol is ethanol.

10. The process as claimed in claim 1 in which a ratio by weight of condensed stream to gas stream entering the condenser is between 5% and 50%.

11. The process as claimed in claim 1 in which a AA heavy products/AE heavy products ratio by weight is between 3/7 and 9/1.

12. The process as claimed in claim 1 in which at least one polymerization inhibitor is introduced at the partial condenser.

13. The process as claimed in claim 1 in which the thermal cracking reaction takes place in an absence of catalyst.

14. The process as claimed in claim 1 in which the thermal cracking reaction takes place in the presence of a catalyst chosen from sulfuric acid, sulfonic acids, phosphoric acid, zeolites, aluminas, catalysts based on silica and alumina, titanates, bases, alkali metals, alkali metal salts, amines, phosphines, or their combination.
